(19) United States
(12) Patent Application Publication
(10) Pub. No.: US 2008/0058741 A1
(43) Pub. Date: Mar. 6, 2008

(54) Products to efficaciously perform toilet training

(76) Inventors: Andrew M. Long, Appleton, WI (US); Ansley C. Allen, Neenah, WI (US); Eliza Geier Basehoar, Neenah, WI (US); Lorelle M. Scheibe, Appleton, WI (US); William J. Raynor, Appleton, WI (US)

Correspondence Address: KIMBERLY-CLARK WORLDWIDE, INC.;Catherine E. Wolf, 401 NORTH LAKE STREET, NEENAH, WI, 54956, US

(21) Appl. No.: 11/511,573

(22) Filed: Aug. 29, 2006

(51) Int. Cl. A61F 13/15

(52) U.S. Cl. 604/361

(57) ABSTRACT

A toilet training system is provided that exhibits an average improvement in Weighted Progress Scale score over baseline of greater than 1.0 over four weeks. Also provided is a method for improving the efficacy of toilet training, the method including producing a toilet training system including an absorbent article having a reactive component, the toilet training system enabling a statistically significant improvement in average Weighted Progress Scale score over baseline over three weeks as compared to use of a substantially identical absorbent article without the reactive component. Also provided is a toilet training system yielding a measurable reduction in time to progress toilet training as compared to use of a wetness liner training pant, wherein the measurable reduction in time is met by achieving the same average Weighted Progress Scale score over baseline in a shorter time period.

### BACKGROUND OF THE INVENTION

The present invention relates to methods for improving the effectiveness of toilet training. More particularly, the present invention pertains to methods for improving the effectiveness of a child's toilet training system and for comparing the effectiveness of various toilet training systems.

Little boys and girls normally wear diapers until they are ready for the toilet training process, when they learn to use the toilet by themselves. The point at which a child will begin this training process is dependent upon many factors, some of which are psychological, some physiological, and some unique to the individual child or their environment.

The toilet training process has been studied and found to encompass multiple stages, ranging from early stages of training characterized by behaviors such as understanding potty words to late stages of training characterized by behaviors such as using a regular toilet without a potty seat. As part of the toilet training process, the parent or caregiver desirably provides instruction and positive encouragement and/or reinforcement that the child should now be using a toilet, instead of diapers. The use of training pants is desirable at some point in the process because it represents a change from diapers to the way that grownups dress and use the toilet.

The toilet training process is complicated by the fact that the successes that a child achieves at any particular stage may also depend upon many factors. These too can by psychological, physiological, or related to the individual child or their environment. Unfortunately, if the child does not respond to an initial toilet training instruction or introduction, the parent or caregiver can be at a loss for identifying more suitable products, training aids, or techniques.

Many absorbent articles have been attempted for use in a training program, such as toilet training or enuresis control, or to provide indication of various medical, physical, or other conditions. Accordingly, various types of sensors and indicators, including moisture or wetness indicators, have been suggested for use in absorbent articles. What is needed in the art are products that improve the effectiveness of a child's toilet training system, and concomitantly a method for proving the effectiveness of various toilet training products.

### SUMMARY OF THE INVENTION

In response to the above-referenced deficiencies in the art, products have been developed for improving the effectiveness of a child's toilet training system. The effectiveness of such products is proven using a progress scale to evaluate a child's changing levels or stages of toilet training.

As caregivers undertake training, indication, and/or notification programs to address toilet training, enuresis control, incontinence monitoring, or condition monitoring, which apply across demographics, those caregivers would benefit by gaining greater access to products that are proven to enhance the effectiveness of such programs.

The invention described herein solves the problems described above and provides an increase in the efficacy of using wetness and other sensors in absorbent articles. In general, the present disclosure is directed to absorbent articles used to sense wetness. The signaling device, for instance, may be configured to indicate to a user that a body fluid is present in the sensing absorbent article.

For example, in one aspect of the present invention, the invention includes a toilet training system exhibiting an average improvement in Weighted Progress Scale score over baseline of greater than 1.0 over four weeks.

In another aspect of the present invention, the invention includes a method for improving the efficacy of toilet training, the method including producing a toilet training system including an absorbent article having a reactive component, the toilet training system enabling a statistically significant improvement in average Weighted Progress Scale score over baseline over three weeks as compared to use of a substantially identical absorbent article without the reactive component.

In another aspect of the present invention, the invention includes a method including producing a toilet training system including an absorbent article having a reactive component, the toilet training system enabling an improvement in average Weighted Progress Scale score over baseline of greater than 1.0 over four weeks, wherein the toilet training system includes an informational item.

In another aspect of the present invention, the invention includes a toilet training system yielding a measurable reduction in time to progress toilet training as compared to use of a wetness liner training pant, wherein the measurable reduction in time is met by achieving the same average Weighted Progress Scale score over baseline in a shorter time period.

### BRIEF DESCRIPTION OF THE DRAWINGS

The foregoing and other features and aspects of the present invention and the manner of attaining them will become more apparent, and the invention itself will be better understood by reference to the following description, appended claims and accompanying drawings, where:

FIG. 1 graphically presents average improvement from baseline Weighted Progress Scale scores using absorbent articles for toilet training;

FIG. 2 graphically presents average improvement from baseline Weighted Progress Scale scores using absorbent articles for toilet training by early stage trainers;

FIG. 3 graphically presents average improvement from baseline Weighted Progress Scale scores using absorbent articles for toilet training by middle stage trainers;

FIG. 4 graphically presents average improvement from baseline Weighted Progress Scale scores using absorbent articles for toilet training by late stage trainers;

FIG. 5 illustrates a perspective view of an absorbent article used to obtain the results of FIG. 1.

Repeat use of reference characters in the present specification and drawings is intended to represent the same or analogous features or elements of the present invention. The drawings are representational and are not necessarily drawn to scale. Certain proportions thereof may be exaggerated, while others may be minimized.

### DETAILED DESCRIPTION OF THE INVENTION

It is to be understood by one of ordinary skill in the art that the present discussion is a description of exemplary aspects of the present invention only, and is not intended as limiting the broader aspects of the present invention.

As used herein, the term “toilet training system” refers to a combination of toilet training garments, toilet training aids, informational items, and behavioral techniques that are used to effect toilet training. The term “toilet training garments” includes disposable diapers, disposable training pants, plastic pants, cloth training pants, and cloth underwear.

The term “toilet training aids” includes potty training story books for children; potty training guide books for parents; potty training videotapes and DVDs for children or parents; progress charts with stickers; stickers alone as rewards; potty training dolls; miniature toilets; potty training flash cards; potty training tip sheets; potty training brochures and pamphlets; dolls and other demonstration devices; reward items such as stickers, crayons, candy, toys, and the like; urine targets; potty training diplomas; starter kits containing a combination of these items; potty chairs; musical potty chairs; wetness awareness devices such as musical alarms, “feel wet” liners, plastic pants, and the like; and toilet paper with children's graphics.

The term “behavioral techniques” includes demonstrating how one uses the toilet; reminding a child to use the potty; limiting or keeping track of a child's intake of fluids; running water while a child is seated; praising for toileting progress; providing cloth training pants; providing disposable training pants; providing potty seat/chair; letting a child decide when to go; not allowing diapers once training is started; giving rewards for success; using a firm and consistent approach; verbal teaching and explanation of toilet training; dressing a child in few/no clothes; disciplining a child; providing children's underwear/panties; placing on potty/toilet at certain intervals; monitoring a child's behavior; having a child sit on the potty for a specified time period; using older children as role models (home or daycare); encouraging a child to be a “Big Kid”; and using toilet training videos and books to motivate a child.

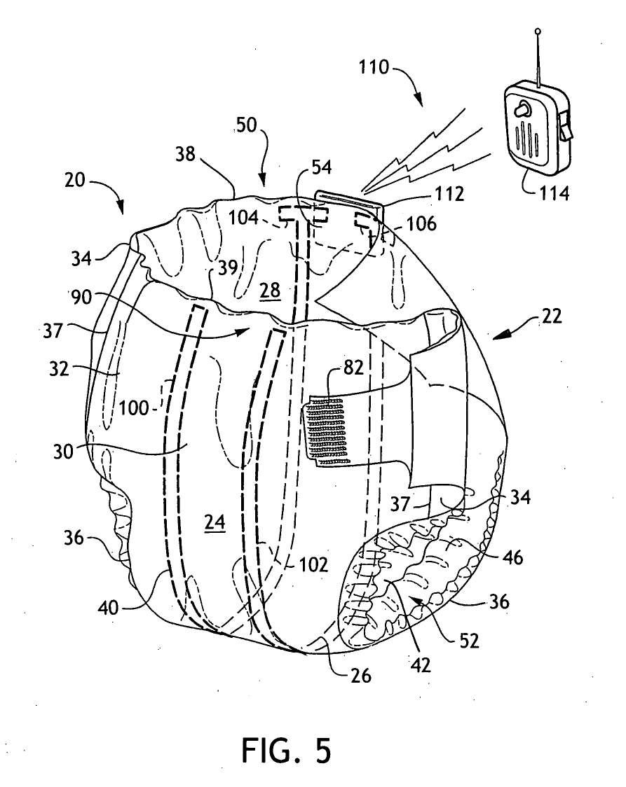

The term “signaling training pants” refers to disposable training pants of the type described below and illustrated in FIG. 5.

The term “reactive component” refers to a component in an absorbent article that reacts to the presence of urine including that from a urine insult. Non-limiting examples include the conductive element(s) in a signaling training pant, the temperature-changing element of a temperature-change training pant, and the sensing component of a sensing absorbent article.

The term “progress scale” refers to any method of assessing a child's toilet training status using a set of two or more ratings whose values are combined to provide a numeric score or numeric scores that measure a child's current state.

A toilet training progress scale and method useful for the present invention is described in co-assigned U.S. Pat. No. 6,250,929 entitled “Methods for Improving the Effectiveness of Toilet Training,” issued to Kolb et al., which is incorporated herein by reference to the extent it is consistent (i.e., not in conflict) herewith.

The toilet training progress scale described therein is used to assess a child's toilet training state at a particular time, and may be used at different times to measure improvement and/or regression during the toilet training process. In one aspect, the progress scale includes a series of questions about learned skills related to toilet training. A parent or caregiver, hereinafter simply referred to as a caregiver, responds to specific questions about the child's toilet training progress. The questions about learned skills may concern, for example, the frequency and location of urination or bowel movements (BM), use of the bathroom, understanding of toilet training terms, activities handled independently by the child, and communications from the child about the need to use the bathroom.

In one particular aspect of the present invention, the toilet training progress scale includes a Weighted Progress Scale (WPS), which is a tool specifically designed to objectively measure a child's toilet training progress. The WPS was developed using a database of information collected during a 2-year longitudinal study conducted in cooperation with the Medical College of Wisconsin (MCOW). The database contains a wealth of information on all the individual behaviors that children must learn on the way to becoming toilet trained. This was accomplished by designing a survey specifically for this purpose. The survey, which was completed weekly during the study by the child's caregiver, collected data on 32 separate toileting behaviors. Four of the items were measures of daily successful use of the potty chair or toilet. The items were rated using either a 4 or 5 point scale corresponding to the number of times, on average, the child exhibited each behavior on a daily basis. The other 28 items assessed a complete array of behaviors associated with learning to use the bathroom successfully. How often the child performed each of these behaviors was rated by the mother using a 1-5 rating scale with 1 representing “never” and 5 representing “always.” Data was collected for up to 16 months on 265 children as they progressed from a pre-training stage through toilet training to a stage of toileting independence.

Each question includes a response format having a plurality of response values, such as “yes” or “no.” The response values may be textual in nature but are desirably assigned a numerical value, such as 1 for “yes” and 0 for “no.” Desirably, the response format for each question includes 3 or more response values. An example of a response format with 3 response values is “never,” “sometimes,” and “always,” which may be assigned numerical values of −1, 0 and 1; 0, 1 and 2; 0, 3 and 5; or the like. Including a greater number of response values allows the strength of response to be measured, for example with questions such as whether a child knows how to urinate in the potty, and allows a finer gradation of frequencies to be measured, for example with questions such as how many times per day does a child sit on the toilet.

The result of the progress scale is a “toilet training progress value” that represents the cumulative value of each of the question response values. The toilet training progress value may consist simply of the sum or the average of the individual question response values. Alternately, the response values are differentially weighted depending upon the significance of the relationship between toilet training and the subject of the question. The differentially-weighted response values may then be added together or averaged to generate the toilet training progress value. As used herein, the terms “cumulative” and “cumulatively” refer to combining the question response values to obtain the toilet training progress value; they are not limited to a specific mathematical approach for combining the response values.

The range of possible resulting values using the progress scale can be divided into a plurality of sub-ranges that represent various stages of toilet training. By way of illustration, the lowest third of the range of possible resulting values may represent the early stages of toilet training, the highest third of the range of possible resulting values may represent the later or final stages of toilet training, and the middle third of the range of possible resulting values may represent the intermediate stages of toilet training. Alternatively, a greater number of sub-ranges may be used to represent a greater number of stages of toilet training.

The toilet training progress value represents a concrete and tangible result that can be used for several useful outcomes including evaluating the present stage of toilet training; comparing the effectiveness of two or more different toilet training methods; assessing the performance of different toilet training aids; and guiding feedback to caregivers for assisting in the toilet training process, including tips and techniques that are likely to be effective at that stage.

For the purposes of the present invention, the methods described above are used to compare the effectiveness of two or more toilet training systems. For instance, the progress scale was used as a measurement tool for assessing the efficacy of training aid improvements. Test protocols were implemented to compare a test treatment group using a training pant with potentially enhanced training features such as a reactive component to a control treatment group using only conventional training pants. By measuring the training status of both groups before and after use, one can statistically determine if the test treatment group has made significantly more progress than the control treatment group.

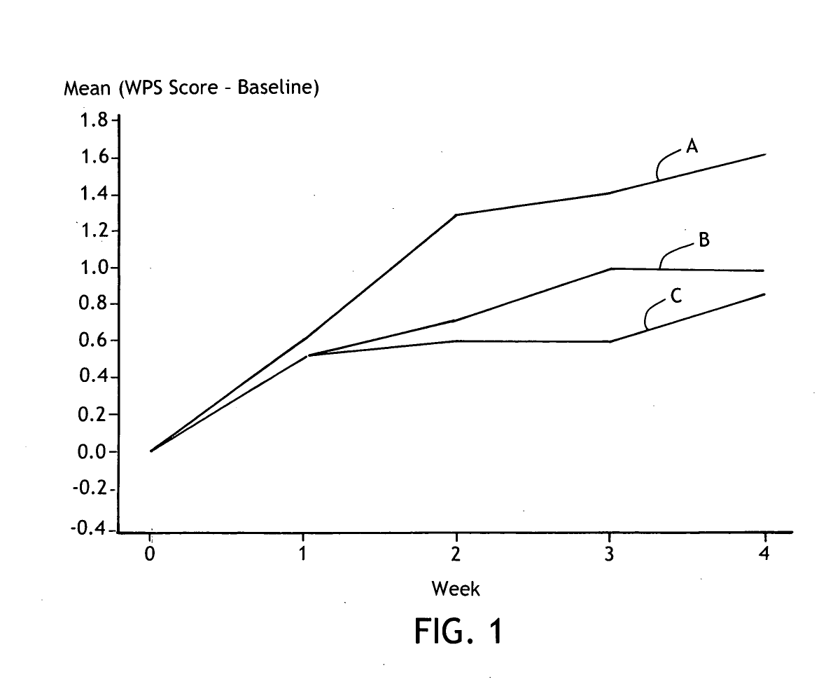

With respect to the present invention, the control treatment group used conventional training pants of the type marketed as HUGGIES PULL-UPS training pants, manufactured in August 2005, and referred to as “C” in FIGS. 1-4. Such conventional training pants are constructed as described below with the exception that the conventional training pants do not include a reactive component. A first test treatment group used newly-developed signaling training pants with enhanced training features of the type described below, manufactured in September 2005, and referred to as “A” in FIGS. 1-4. A second test treatment group used commercially-available PAMPERS FEEL 'N LEARN wetness liner training pants, purchased in July 2005, and referred to as “B” in FIGS. 1-4.

Potential participants were recruited with a goal of achieving fifty participants per treatment group. Potential participants were initially screened based on stage of toilet training and randomly assigned to treatment groups to achieve a balance of different stages in the different treatment groups. A WPS evaluation was completed for each participant at the beginning of the study to establish a baseline and after each week of the study. Potential participants whose initial WPS score was greater than 4 were dropped from further analyses because such participants were daytime-trained according to the scale. Participants who did not answer all WPS questions at the initial placement date did not have a baseline and were dropped from further analyses.

The study collected data on 32 separate toileting behaviors. Four (4) of the items were measures of daily successful use of the potty chair or toilet. The items were rated using either a 4 or 5 point scale (detailed below) corresponding to the number of times, on average, the child was doing each behavior on a daily basis. The other 28 items assessed a complete array of behaviors associated with learning to use the bathroom successfully. How often the child performed each of these behaviors was rated by the mother using a 1-5 rating scale with 1 representing “never” and 5 representing “always.”

The measures of daily success used in the WPS are listed below. The first three of these questions had a response format with five response values (0, 1-2, 3-5, 6-8, and 9+), and the fourth question had a response format with four response values (0, 1, 2, and 3+).

On Average, How Many Times a Day did Your Child . . .
1) Urinate (pee) or have a BM (poop) in their pants/diapers?
2) Sit on the toilet or potty seat/chair?
3) Urinate (pee) in the toilet or potty chair?
4) Have a BM (poop) in the toilet or potty chair?

The other 28 WPS questions are listed below. All of these questions had a response format with five response values (1, 2, 3, 4, and 5), where response value 1 was coded “Never,” response value 3 was coded “Sometimes,” and response value 5 was coded “Always.”

Select a Response Value Corresponding to Level Your Child is at This Week:
1) Stays dry for over two (2) hours
2) Has regular bowel movements (approximately same time daily)
3) Has a potty chair or potty seat available (in addition to a toilet)
4) Understands potty words (like pee, wee wee, BM, potty, etc.)
5) Indicates a physical need to go potty by wiggling, pausing, hiding, etc.
6) Wears a training pant or underwear
7) Sits on the potty when placed (for 5 minutes)
8) Shows an interest in using the potty by talking, pointing, watching
9) Tells you during or after a urine (pee) accident
10) Tells you during or after a bowel movement (BM, poop)
11) Pulls training pants or underwear up by him/herself
12) Pulls training pants or underwear down by him/herself
13) Urinates in the potty with your help
14) Flushes toilet by him/herself
15) Wipes urine (pee) effectively by herself (GIRLS ONLY)
16) Urinates (pees) while standing by himself (BOYS ONLY)
17) Wipes BM (poop) effectively by him/herself
18) Washes hands by him/herself
19) Tells you before having a bowel movement (poop)
20) Tells you before having to urinate (pee)
21) Knows how to urinate (pee) in the potty
22) Enters bathroom and urinates (pees) in the potty by him/herself
23) Enters bathroom and has a BM (poop) in the potty by him/herself
24) Stays dry during the day
25) Stays BM (poop) free during the day
26) Wakes up BM (poop) free overnight
27) Wakes up dry overnight
28) Uses the regular toilet without potty seat

In this particular study, the resulting equations for girls and boys were:

WPS Girls=(−7.451039547)+(0.128152172) (rating item 1)+(0.041971246) (rating item 2)+(0.239076402) (rating item 3)+(0.145284682) (rating item 4)+(−0.001671888) (rating item 5)+(0.038417748) (rating item 6)+(−0.060396761) (rating item 7)+(0.020772648) (rating item 8)+(0.246372924) (rating item 9)+(−0.004745736) (rating item 10)+(0.061745678) (rating item 11)+(0.060004417) (rating item 12)+(0.017147363) (rating item 13)+(0.060518663) (rating item 14)+(0.302706042) (rating item 15)+(0.048527553) (rating item 16)+(0.103962941) (rating item 17)+(0.09893343) (rating item 18)+(0) (rating item 19)+(0.014404783) (rating item 20)+(0.162216406) (rating item 21)+(0.073733072) (rating item 22)+(0.181284268) (rating item 23)+(0.163142281) (rating item 24)+(0.16158228) (rating item 25)+(0.097555822) (rating item 26)+(0.37625208) (rating item 27)+(0.125265405) (rating item 28)+(0.003272056) (rating item 29)+(0.083625853) (rating item 30)+(0.065309514) (rating item 31).

WPS Boys=(−6.951455)+(0.084994649) (rating item 1)+(−0.04725484) (rating item 2)+(0.182468882) (rating item 3)+(0.289820732) (rating item 4)+(0 086541745) (rating item 5)+(0.033689299) (rating item 6)+(−0.01672961) (rating item 7)+(0.017864443) (rating item 8)+(0.160919483) (rating item 9)+(0.08221516) (rating item 10)+(0.11073007) (rating item 11)+(0.089483695) (rating item 12)+(0.006554928) (rating item 13)+(0.04290046) (rating item 14)+(0.242002886) (rating item 15)+(0.049427276) (rating item 16)+(0.03412295) (rating item 17)+(0) (rating item 18)+(0.090071031) (rating item 19)+(−0.14351426) (rating item 20)+(0.056435169) (rating item 21)+(0.132503394) (rating item 22)+(0.120012498) (rating item 23)+(0.393357222) (rating item 24)+(0.192515585) (rating item 25)+(0.164606049) (rating item 26)+(0.186182297) (rating item 27)+(0.050362164) (rating item 28)+(−0.02472152) (rating item 29)+(0.032205617) (rating item 30)+(0.246386367) (rating item 31).

Analyses were conducted using the change from baseline WPS scores for each participant as the primary response. For the results described herein, a large negative score corresponds to a regression in toilet training. A large positive score corresponds a great deal of progress or completion of toilet training.

Results of the analyses are shown in FIG. 1, which illustrates the average improvement from baseline WPS scores for each treatment group. For the training pant products, the average increase in WPS score from baseline is significantly greater at a two-sided 90% or greater confidence level for children wearing signaling training pants than for children wearing conventional training pants or for children wearing PAMPERS FEEL 'N LEARN wetness liner training pants. The average increases in WPS score from baseline for children wearing conventional training pants and for children wearing PAMPERS FEEL 'N LEARN wetness liner training pants are not statistically different.

For example, use of signaling training pants exhibits an average improvement in Weighted Progress Scale score over baseline of greater than 1.0, greater than 1.2, and greater than 1.4 over four weeks. Use of signaling training pants exhibits an average improvement in Weighted Progress Scale score over baseline of greater than 1.0 over three weeks and over two weeks.

Looking at the results in a different way, use of signaling training pants achieves an average improvement in Weighted Progress Scale score over baseline of greater than 0.9 one week sooner than by use of PAMPERS FEEL 'N LEARN wetness liner training pants. Use of the signaling training pants yields a measurable reduction in time to achieve a given progress in toilet training as compared to use of PAMPERS FEEL 'N LEARN wetness liner training pants. In one example, the measurable reduction in time is met by achieving the same average Weighted Progress Scale score over baseline in a shorter time period.

The treatment groups were compared using a repeated measures analysis of covariance. The covariates included gender of the child, the week of the measurement, and each child's stage in training at the beginning of the study. Stage in training was defined as early stage when the initial WPS score was from −4 up to −1, middle stage when the initial WPS score was from −1 up to +1, and late stage when the initial WPS score was from +1 up to +4.

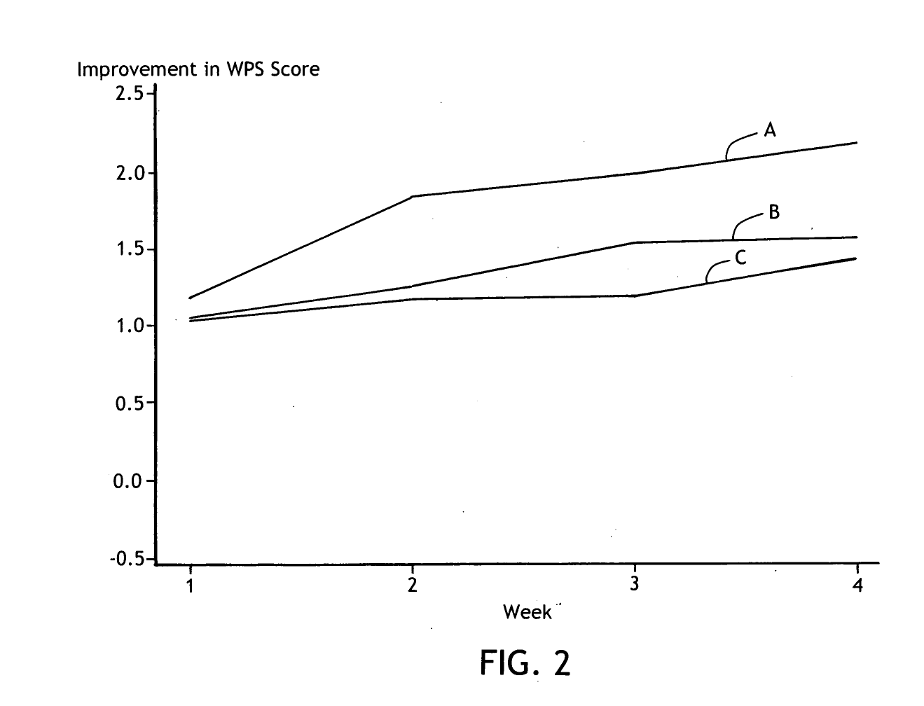

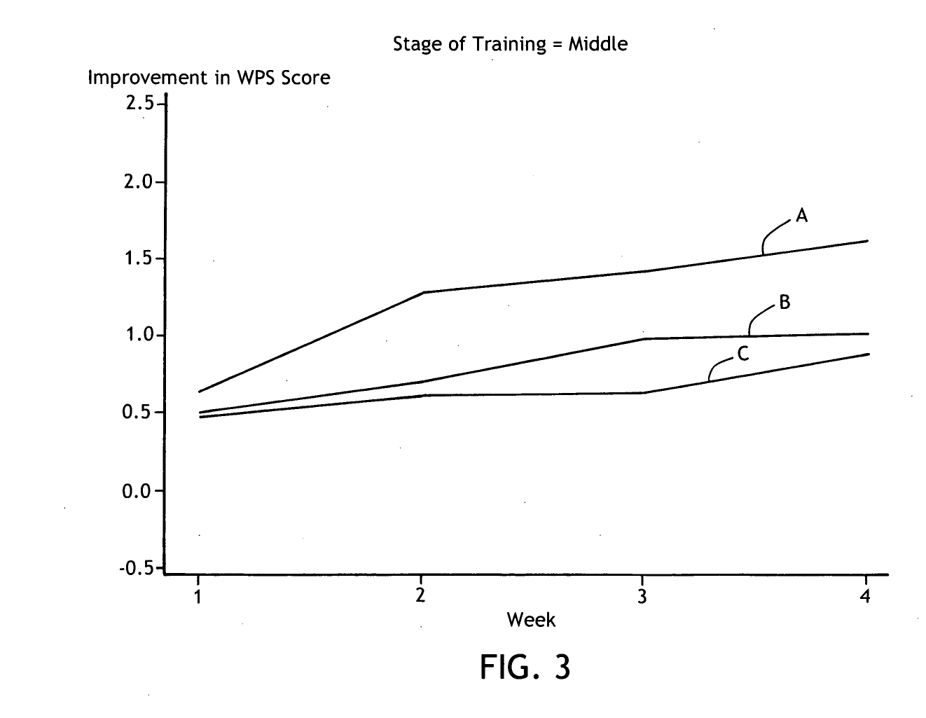

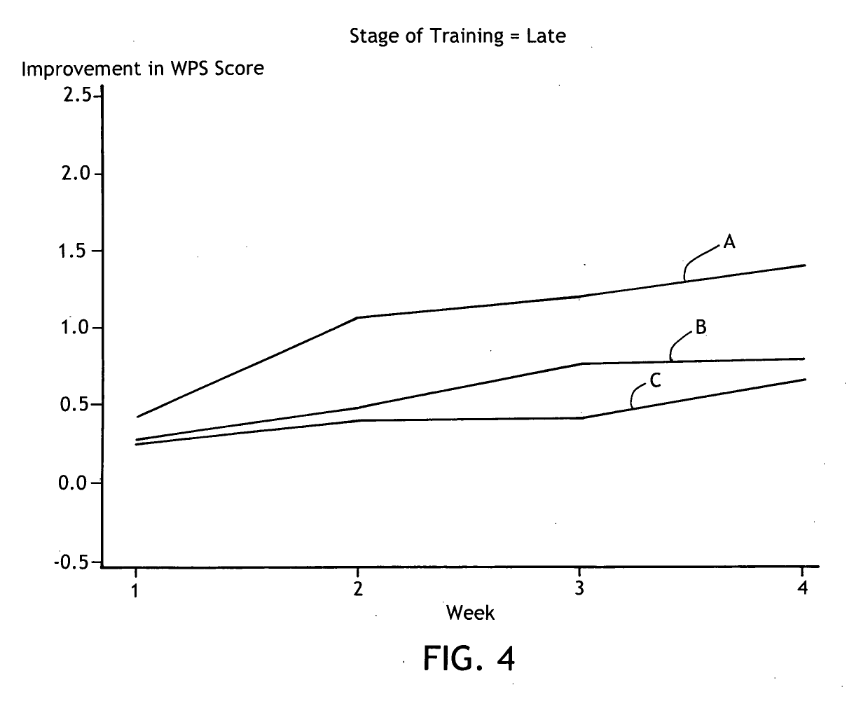

FIGS. 2-4 illustrate the predicted improvements in WPS scores at each stage in training. FIG. 2 shows the predicted improvement in WPS scores for early stage trainers. FIG. 3 shows the predicted improvement in WPS scores for middle stage trainers. FIG. 4 shows the predicted improvement in WPS scores for late stage trainers.

For example, use of signaling training pants exhibited an average improvement in Weighted Progress Scale score over baseline is greater than 1.5, greater than 1.6, and greater then 1.7 for early stage trainers. The use of signaling training pants exhibited an average improvement in Weighted Progress Scale score over baseline is greater than 1.0, greater than 1.2, and greater than 1.4 for middle stage trainers. The use of signaling training pants exhibited an average improvement in Weighted Progress Scale score over baseline is greater than 0.8, greater than 1.0, and greater than 1.2 for late stage trainers.

Over all weeks, early stage trainers make significantly more progress towards potty training than middle and late stage trainers at a 0.05 significance level. For the training pant products, the predicted improvement in WPS score is significantly greater for children wearing signaling training pants than for children wearing conventional training pants or for children wearing PAMPERS FEEL 'N LEARN wetness liner training pants.

The present disclosure is generally directed to training absorbent articles, the use of which improves toilet training. One exemplary type of training absorbent article is a sensing absorbent article adapted to be attached to a signaling device that may be configured to indicate the presence of a body fluid in the absorbent article, otherwise referred to herein as a signaling training pant.

In the type of sensing article used herein, wetness sensing absorbent articles include an open circuit that becomes closed when a conductive fluid, such as a body fluid, is present in between a pair of conductive leads. The wetness sensing absorbent articles containing the circuit are disposable meaning that they are designed to be discarded after a limited use rather than being laundered or otherwise restored for reuse.

The circuit contained within the wetness sensing absorbent articles of the present disclosure is configured to be attached to a signaling device. The signaling device provides power to the circuit while also including an audible signal that indicates to the user the presence of a body fluid. Although the wetness sensing absorbent article may itself be disposable, the signaling device is reusable from article to article.

Referring to FIG. 5, for non-limiting exemplary purposes, a wetness sensing absorbent article 20 is shown in a partially fastened condition.

Various materials and methods for constructing absorbent articles are disclosed in U.S. Pat. No. 4,798,603 issued Jan. 17, 1989, to Meyer et al.; U.S. Pat. No. 5,176,672 issued Jan. 5, 1993, to Bruemmer et al., U.S. Pat. No. 5,509,915 issued Apr. 23, 1996 to Hanson et al., U.S. Pat. No. 5,993,433 issued Nov. 30, 1999 to St. Louis et al., and U.S. Pat. No. 6,248,097 issued Jun. 19, 2001 to Beitz et al., PCT Patent Application WO 00/37009 published Jun. 29, 2000 by A. Fletcher et al; U.S. Pat. No. 4,940,464 issued Jul. 10, 1990 to Van Gompel et al.; U.S. Pat. No. 5,766,389 issued Jun. 16, 1998 to Brandon et al., and U.S. Pat. No. 6,645,190 issued Nov. 11, 2003 to Olson et al. which are incorporated herein by reference to the extent they are consistent (i.e., not in conflict) herewith. The products disclosed in these references are provided as examples of materials and construction methods and do not inherently exhibit the parameters claimed herein.

The wetness sensing absorbent article 20 defines a pair of longitudinal end regions, otherwise referred to herein as a front region 22 and a back region 24, and a center region, otherwise referred to herein as a crotch region 26, extending longitudinally between and interconnecting the front and back regions 22, 24. The wetness sensing absorbent article 20 also defines an inner surface 28 adapted in use (e.g., positioned relative to the other components of the article 20) to be disposed toward the wearer, and an outer surface 30 opposite the inner surface. The front and back regions 22, 24 are those portions of the wetness sensing absorbent article 20, which when worn, wholly or partially cover or encircle the waist or mid-lower torso of the wearer. The crotch region 26 generally is that portion of the wetness sensing absorbent article 20 which, when worn, is positioned between the legs of the wearer and covers the lower torso and crotch of the wearer. The wetness sensing absorbent article 20 has a pair of laterally opposite side edges 36 and a pair of longitudinally opposite waist edges, respectively designated front waist edge 38 and back waist edge 39.

The illustrated wetness sensing absorbent article 20 includes a chassis 32 that encompasses the front region 22, the back region 24, and the crotch region 26. The chassis 32 includes an outer cover 40 and a bodyside liner 42 that is joined to the outer cover 40 in a superimposed relation therewith by adhesives and ultrasonic bonds. The liner 42 is disposed toward the wearer's skin during wear of the wetness sensing absorbent article 20. The chassis 32 further includes an absorbent structure (not shown) disposed between the outer cover 40 and the bodyside liner 42 for absorbing liquid body exudates exuded by the wearer, and further includes a pair of containment flaps 46 secured to the bodyside liner 42 for inhibiting the lateral flow of body exudates. Suitable constructions and arrangements for the containment flaps 46 are generally well known to those skilled in the art and are described in U.S. Pat. No. 4,704,116 issued Nov. 3, 1987 to Enloe, which is incorporated herein by reference.

To further enhance containment and/or absorption of body exudates, the wetness sensing absorbent article 20 may also include leg elastic members (not shown), as are known to those skilled in the art.

As shown in FIG. 5, the wetness sensing absorbent article 20 further includes a pair of opposing elastic side panels 34 that are attached to the back region of the chassis 32. The side panels 34 may be stretched around the waist and/or hips of a wearer to secure the garment in place. The elastic side panels are attached to the chassis along a pair of opposing longitudinal edges 37. The side panels 34 may be attached or bonded to the chassis 32 using any suitable bonding technique. Ultimately, the side panels 34 are generally aligned with a waist region 90 of the chassis.

The side panels 34 are connected to the back region of the wetness sensing absorbent article 20 and extend over the front region of the article when securing the article in place on a user.

With the wetness sensing absorbent article 20 in the fastened position as partially illustrated in FIG. 5, the elastic side panels 34 are connected by a fastening system 82 to define a 3-dimensional wetness sensing absorbent article configuration having a waist opening 50 and a pair of leg openings 52. The waist opening 50 of the article 20 is defined by the waist edges 38 and 39 which encircle the waist of the wearer.

In the aspects of the present invention shown in FIG. 5, the side panels are releasably attachable to the front region 22 of the article 20 by the fastening system 82.

The fastening system 82 includes laterally opposite first fastening components adapted for refastenable engagement to corresponding second fastening components. The fastening components include mechanical fastening elements for improved performance.

The wetness sensing absorbent article 20 includes waist elastic members for providing elasticity around the waist opening. As shown in the FIG. 5, the wetness sensing absorbent article 20 includes a front waist elastic member 54.

As described above, the present disclosure is particularly directed to incorporating a body fluid indicating system. One such system is described below. Other systems include a wetness liner such as that described in U.S. Pat. No. 6,658,432 to Underhill et al., a temperature system, a system in which graphics fade or appear, and any other suitable body fluid indicating system.

One such body fluid indicating system is the wetness indicating system described herein. In this regard, as shown in FIG. 5, the wetness sensing absorbent article 20 includes a first conductive element 100 spaced from a second conductive element 102. In this aspect of the present invention, the conductive elements extend from the front region 22 of the wetness sensing absorbent article to the back region 24 without intersecting. The conductive elements 100 and 102 include a conductive foil such as that described in U.S. Pat. No. 6,417,455 issued Jul. 9, 2002 to Zein et. Al. The first conductive element 100 may not intersect the second conductive element 102 in order to form an open circuit that may be closed, for instance, when a conductive fluid is positioned in between the conductive elements. In other aspects of the present invention, however, the first conductive element 100 and the second conductive element 102 may be connected to a sensor within the chassis. The sensor may be used to sense changes in temperature or may be used to sense the presence of a particular substance, such as a metabolite.

In the aspect of the present invention shown in FIG. 5, the conductive elements 100 and 102 extend the entire length of the wetness sensing absorbent article 20. It should be understood, however, that in other aspects of the present invention the conductive elements may extend only to the crotch region 26 or may extend to any particular place in the wetness sensing absorbent article where a body fluid is intended to be sensed.

The conductive elements 100 and 102 may be incorporated into the chassis 32 at any suitable location as long as the conductive elements are positioned so as to contact a body fluid that is absorbed by the wetness sensing absorbent article 20. In this regard, the conductive elements 100 and 102 generally lie inside the outer cover 40. In fact, in one aspect of the present invention, the conductive elements 100 and 102 may be attached or laminated to the inside surface of the outer cover 40 that faces the absorbent structure. Alternatively, however, the conductive elements 100 and 102 may be positioned on the absorbent structure or positioned on the liner 42.

The conductive element 100 and 102 are connected directly to a signaling device. The first conductive element 100 is attached to a first conductive pad member 104, while the second conductive element 102 is connected to a second conductive pad member 106. The pad members 104 and 106 are provided for making a reliable connection between the open circuit formed by the conductive elements to a signaling device that is intended to be installed on the chassis by the consumer or manufacturer. The pad members 104 and 106 create a target zone for attaching the signaling device and the conductive leads or elements.

Further details related to the structure, features, and materials and potential alternatives thereof of the absorbent article 20 may be found in co-pending and co-assigned U.S. patent application Ser. No. 11/414,032, filed on Apr. 27, 2006 by Allen, et al. and titled “An Array of Wetness Sensing Articles”; which is incorporated herein by reference to the extent it is consistent (i.e., not in conflict) herewith.

Referring to FIG. 5, a signaling device 110 (as depicted by ref. numerals 112 and 114) is shown attached to the conductive pad members 104 and 106. As shown, in this aspect of the present invention, the signaling device generally 110 includes a transmitter 112. In other aspects of the present invention, the signaling device 110 may also include a receiver 114. The transmitter 112 includes a pair of opposing terminals that are electrically connected to the corresponding conductive elements. When a body fluid is present in the wetness sensing absorbent article 20, the open circuit formed by the conductive elements 100 and 102 is closed which, in turn, activates the signaling device 110. In particular, in this aspect of the present invention, the transmitter 112 broadcasts an audible alarm or musical selection. In other aspects of the present invention, the transmitter 112 sends a wireless signal to the receiver 114 which then indicates to a user that a body fluid is present in the wetness sensing absorbent article 20.

The signaling device 110 can emit an audible signal or a visual signal in order to indicate to the user that the circuit has been closed. The audible signal, for instance, may be as simple as one or more beeps to perhaps emitting a musical tune. Similarly, if the signaling device 110 issues a visible signal, the visible signal may comprise a few lights or an interactive display. In still another aspect of the present invention, the receiver 114 of the signaling device 110 may be configured to vibrate when the circuit within the wetness sensing absorbent article is closed.

In one aspect of the present invention, the signaling device 110 includes a transmitter 112 in combination with a receiver 114. It should also be understood, however, that the signaling device may comprise a single unit that remains attached to the wetness sensing absorbent article 20. For example, the signaling device may be mounted on the wetness sensing absorbent article and issue a visible signal and/or an audible signal from the article itself.

In various other aspects of the present invention, the wetness sensing absorbent article 20 may include additional features such as those disclosed in co-pending and co-assigned U.S. patent application Ser. No. 11/303,283, filed Dec. 15, 2005 by Long, et al. and entitled “Garments With Easy-To-Use Signaling Device”; and U.S. patent application Ser. No. 11/215,937, filed Aug. 31, 2005 by Ales, et al. and entitled “Method of Detecting the Presence of an Insult in an Absorbent Article and Device for Detecting the Same”; which are incorporated herein by reference to the extent they are consistent (i.e., not in conflict) herewith. For example, the wetness sensing absorbent article may also include other wetness sensing features such as fading ink, appearing ink, a wetness liner, or a cooling component.

The wetness sensing absorbent article 20 may be a part of a wetness sensing system such as those described in co-pending and co-assigned U.S. patent application Ser. No. 11/414,032, filed on Apr. 27, 2006 by Allen, et al. and titled “An Array of Wetness Sensing Articles”; which is incorporated herein by reference to the extent it is consistent (i.e., not in conflict) herewith.

In various aspects of the present invention, the wetness sensing system may include informational items such as instructions in the use of the product and tips for toilet training, enuresis control, or incontinence control. As used herein, the term “informational item” refers to objects that are provided in addition to training articles, are adapted to communicate information to the user and/or consumer of the training article, and are associated with individual components of the system. Examples of informational items include cards, paper, electronic media, printing on the packaging, or other suitable media capable of storing and conveying information.

In various aspects, the informational items associated with the system components may be adapted to appeal to the specific category of user and/or purchaser to which the training article is adapted. The informational items may be adapted, for example, by providing information likely to be of interest to a given category of user and/or purchaser.

For example, a training article may be adapted for use by a caregiver for toilet training purposes. An informational item may be associated with the training article that is adapted to interest caregivers. For example, the informational item may be a card containing information or instructions about children's health and hygiene, such as sleep habits, thumb sucking, teething, skin health, toilet training; questions to ask a child; jokes; and the like, and combinations thereof. The informational item may additionally or alternatively include addresses for web sites available on the Internet. The web sites may contain information related to issues of interest for caregivers and users of training articles.

The informational item may additionally or alternatively include information describing activities that are suitable for caregivers and users of training articles. The activities may be adapted for a child at a specific age, size, and/or stage of development. For example, the activities may be adapted to promote interaction between the child and the caregiver.

The informational item may additionally or alternatively include information describing the benefits to be derived from using the system. This informational item would be part of a promotional plan emphasizing the customizability of the system for the benefit of the consumer, caregiver, and/or user. This informational item would both explain the use of the various components of the system as well as present the additional components that may be available and the various combinations that are possible to achieve different goals.

These and other modifications and variations to the present invention may be practiced by those of ordinary skill in the art, without departing from the spirit and scope of the present invention, which is more particularly set forth in the appended claims. In addition, it should be understood that aspects of the various aspects of the present invention may be interchanged either in whole or in part. Furthermore, those of ordinary skill in the art will appreciate that the foregoing description is by way of example only, and is not intended to limit the invention so further described in such appended claims.

1. A toilet training system exhibiting an average improvement in Weighted Progress Scale score over baseline of greater than 1.0 over four weeks, the toilet training system including an absorbent article, an informational item, and a behavioral technique.

2. The system of claim 1, wherein the average improvement in Weighted Progress Scale score over baseline is greater than 1.2.

3. The system of claim 1, wherein the average improvement in Weighted Progress Scale score over baseline is greater than 1.4.

4. The system of claim 1, wherein the average improvement in Weighted Progress Scale score over baseline is over three weeks.

5. The system of claim 1, wherein the average improvement in Weighted Progress Scale score over baseline is over two weeks.

6. The system of claim 1, wherein the average improvement in Weighted Progress Scale score over baseline is greater than 1.5 for early trainers, where early trainers are subjects with an initial WPS score between −4 and −1.

7. The system of claim 1, wherein the average improvement in Weighted Progress Scale score over baseline is greater than 1.6 for early trainers, where early trainers are subjects with an initial WPS score between −4 and −1.

8. The system of claim 1, wherein the average improvement in Weighted Progress Scale score over baseline is greater than 1.7 for early trainers, where early trainers are subjects with an initial WPS score between −4 and −1.

9. The system of claim 1, wherein the average improvement in Weighted Progress Scale score over baseline is greater than 1.0 for middle trainers, where middle trainers are subjects with an initial WPS score between −1 and 1.

10. The system of claim 1, wherein the average improvement in Weighted Progress Scale score over baseline is greater than 1.2 for middle trainers, where middle trainers are subjects with an initial WPS score between −1 and 1.

11. The system of claim 1, wherein the average improvement in Weighted Progress Scale score over baseline Is greater than 1.4 for middle trainers, where middle trainers are subjects with an initial WPS score between −1 and 1.

12. The system of claim 1, wherein the average improvement in Weighted Progress Scale score over baseline is greater than 0.8 for late trainers, where late trainers are subjects with an initial WPS score between 1 and 4.

13. The system of claim 1, wherein the average improvement in Weighted Progress Scale score over baseline is greater than 1.0 for late trainers, where late trainers are subjects with an initial WPS score between 1 and 4.

14. The system of claim 1, wherein the average improvement in Weighted Progress Scale score over baseline is greater than 1.2 for late trainers, where late trainers are subjects with an initial WPS score between 1 and 4.

15. The system of claim 1, further comprising an absorbent article having a reactive component.

16. The system of claim 15 wherein the reactive component includes an electrically-conductive element.

17. The system of claim 15, wherein the reactive component includes a temperature-change element.

18. The system of claim 1, further comprising an absorbent article having a signaling system adapted to indicate the presence of urine.

19. The system of claim 18, wherein the presence of urine is indicated with an audible signal.

20. A method for improving the efficacy of toilet training, the method comprising:
producing a toilet training system including an absorbent article having a reactive component, an informational item, and a behavioral technique, the toilet training system enabling a statistically significant improvement in average Weighted Progress Scale score over baseline over three weeks as compared to use of a substantially identical absorbent article without the reactive component.

21. The method of claim 20, wherein the absorbent article is a training pant.

22. The method of claim 20, wherein the reactive component includes an electrically-conductive element.

23. The method of claim 20, wherein the reactive component includes a temperature-change element.

24. A method comprising:
producing a toilet training system including an absorbent article having a reactive component, an informational item, and a behavioral technique, the toilet training system enabling an improvement in average Weighted Progress Scale score over baseline of greater than 1.0 over four weeks, wherein the toilet training system includes an informational item.

25. A toilet training system yielding a measurable reduction in time to progress toilet training as compared to use of a wetness liner training pant, wherein the measurable reduction in time is met by achieving the same average Weighted Progress Scale score over baseline in a shorter time period, the toilet training system including an absorbent article, an informational item, and a behavioral technique.
